(19) United States
(12) Patent Application Publication
(10) Pub. No.: US 2025/0045918 A1
(43) Pub. Date: Feb. 6, 2025

(54) SYSTEMS AND METHODS FOR FACILITATING ANALYSIS OF ANATOMICAL STRUCTURES

(71) Applicant: Intuitive Research and Technology Corporation, Hunstville, AL (US)

(72) Inventors: Steven Michael Thomas, Madison, AL (US); Cayla Ray Garrett, Hunstville, AL (US); Olivia Grace Weaver, Huntsville, AL (US); Harleigh Marie Bass, Huntsville, AL (US)

(21) Appl. No.: 18/793,554

(22) Filed: Aug. 2, 2024

Related U.S. Application Data
(60) Provisional application No. 63/517,686, filed on Aug. 4, 2023.

(51) Int. Cl. G06T 7/00; G06T 15/08; G06T 17/00; G16H 30/40

(52) U.S. Cl. CPC G06T 7/0012; G06T 15/08; G06T 17/00; G16H 30/40; G06T 2210/41

(57) ABSTRACT

A system for facilitating analysis of anatomical structures is configurable to: (i) access a subject-specific 3D representation of one or more anatomical structures, the subject-specific 3D representation being generated based on a set of 2D images of a subject; (ii) access an idealized 3D representation of the one or more anatomical structures; and (iii) simultaneously display the subject-specific 3D representation and the idealized 3D representation in navigable form within a virtual 3D environment.

### CROSS-REFERENCE TO RELATED APPLICATIONS

This application claims priority to U.S. Provisional Patent Application No. 63/517,686, filed on Aug. 4, 2023, and entitled “SYSTEMS AND METHODS FOR FACILITATING ANALYSIS OF ANATOMICAL STRUCTURES”, the entirety of which is incorporated herein by reference for all purposes.

### BACKGROUND

Various medical imaging modalities exist, such as x-ray, computed tomography (CT), computed tomography perfusion (CTP) imaging, positron emission tomography (PET), single-photon emission computed tomography (SPECT), magnetic resonance imaging (MRI), ultrasound, and/or others. Many medical imaging modalities generate a set of images (referred to as “slices” or “image slices”) that provides representations of structures within a patient's body. The slices of the set of images are typically associated with different positions along a patient's body. For example, where each image depicts a cross-section of the patient's body in the x-dimension and the y-dimension, each image may be associated with a different z-position (e.g., height). In this regard, a subset of contiguous image slices may provide contiguous representations of cross-sections of the patient's body. Structures of a patient's body may thus be depicted in multiple image slices.

The subject matter claimed herein is not limited to embodiments that solve any challenges or that operate only in environments such as those described above. Rather, this background is only provided to illustrate one exemplary technology area where some embodiments described herein may be practiced.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates example components of a system that may comprise or implement the disclosed embodiments;

FIGS. 2A and 2B illustrate an example of a subject-specific 3D representation of anatomical structures of a subject based on a set of 2D images of the subject;

FIGS. 3A, 3B, and 3C illustrate example subject-specific 3D representations of anatomical structures of a subject.

FIGS. 4A and 4B illustrate an example user interface for receiving input directed to changing presentation characteristics of a subject-specific 3D representation of an anatomical structure of a subject.

FIGS. 5A and 5B illustrate an example simultaneous display of a subject-specific 3D representation of an anatomical structures and idealized 3D representations of the anatomical structures.

FIGS. 6A, 6B, 6C, and 6D illustrate example idealized 3D representations of different anatomical structures.

FIGS. 7A, 7B, 7C, and 7D illustrate example use of a highlight sphere tool for interacting with 3D representations of anatomical structures.

FIGS. 8A, 8B, and 8C illustrate example use of a measurement tool for interacting with 3D representations of anatomical structures.

FIGS. 9A, 9B, and 9C illustrate example use of an annotation tool for interacting with 3D representations of anatomical structures.

FIG. 10 illustrates example modification to the positioning of an idealized 3D representation within a virtual environment.

FIGS. 11A and 11B illustrate example resizing of a subject-specific 3D representation of an anatomical structure.

### DETAILED DESCRIPTION

Disclosed embodiments are directed to systems, methods, apparatuses, and techniques for facilitating analysis of anatomical structures.

Medical training programs often involve presenting trainees with idealized models of bodily structures to help trainees develop and/or refine anatomical understanding and/or recognition skills. Such idealized models can be presented in various formats, such as within textbooks, using physical models, or using computer systems (e.g., to enable navigation of an idealized 3D model).

However, many newly trained medical practitioners experience difficulty in transitioning from interpreting idealized anatomical models (e.g., in the educational setting) to interpreting real-patient data representative of real-patient anatomies, such as medical images of patients (e.g., x-ray, CT, CTP, PET, SPECT, MRI, ultrasound, and/or others). For instance, real patients may have anatomical structures that deviate from idealized anatomical structures in various ways that can be difficult for medical trainees to understand or expect. Such deviations can present difficulties for newly trained medical practitioners in diagnostic, treatment, and/or other contexts where medical imagery capturing patient bodily structures is used.

At least some disclosed embodiments are directed to techniques for facilitating analysis of anatomical structures by simultaneously displaying a subject-specific 3D representation and an idealized 3D representation of patient anatomy. The subject-specific 3D representation may be generated based on medical images of a real patient, and the idealized 3D representation may be a manually designed or computer generated 3D model of patient anatomy. An idealized 3D representation may be configured to illustrate aspects of healthy patient anatomy or afflicted patient anatomy (e.g., patient anatomy when one or more pathologies are present). The subject-specific 3D representation and the idealized 3D representation may be displayed within a navigable 3D environment, enabling users to interact with the 3D representations within the same environment.

Simultaneously displaying subject-specific 3D representations in conjunction with idealized 3D representations can assist users in expanding their understanding of idealized patient anatomy (e.g., academic understanding) to obtain an understanding of representations of real patient anatomy (e.g., practical understanding). For instance, the idealized 3D representation may act as a reference that the user may refer to when encountering representations of real patient anatomical structures. Such a reference can enable users to perceive similarities to and/or deviations from idealized anatomy that can occur in real patient care contexts. For example, a trainee may be presented with a 3D model of an idealized, healthy human brain simultaneously with a 3D model of a brain of a real human patient that has experienced a stroke, enabling the user to gain an understanding of the anatomical effects that a stroke can have on a human brain.

The techniques described herein may be implemented in medical training programs to enable users to improve in their ability to interpret medical imagery in real patient care contexts. Training medical practitioners in such a manner may result in medical practitioners with increased intuitive understanding of how to interpret medical imagery, which can improve patient care outcomes.

Although the present description focuses, in at least some respects, on medical training and/or educational contexts, the principles described herein may be implemented in patient care and/or clinical contexts. For instance, a 3D representation of patient anatomy may be used as a basis for identifying an idealized model for presentation to a medical practitioner. The idealized model may be selected from a library of idealized models for various types of patients (e.g., patients with different ages, weights, heights, ethnicities, medical histories, pathologies, etc.). The identified idealized model may provide insight to the medical practitioner of potential pathologies the patient is experiencing.

Although the present description focuses, in at least some respects, on representations of human patients/individuals, the principles described herein may be applied to animals and/or other imaging subjects.

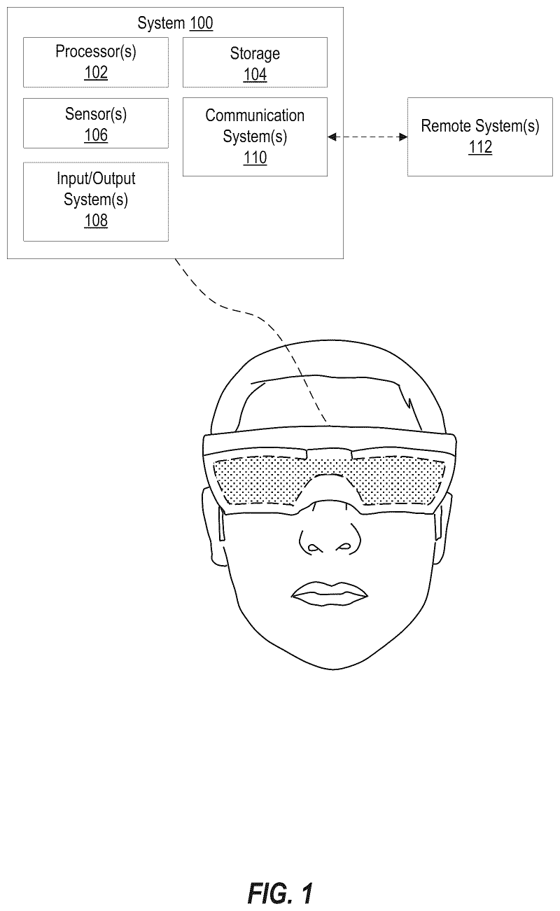

Attention is now directed to FIG. 1, which illustrates an example system 100 that may include or be used to implement one or more disclosed embodiments. FIG. 1 depicts the system 100 as a head-mounted display (HMD) configured for placement over a head of a user to display virtual content for viewing by the user's eyes. Such an HMD may comprise a system configured to provide users with augmented reality (AR) experiences (e.g., with virtual content overlaid on a user's view of the real world), virtual reality (VR) experiences (e.g., immersive experiences where the user's view of the real world is obstructed), and/or any other type of extended reality (XR) experience. Although the present disclosure focuses, in at least some respects, on a system 100 implemented as an HMD, it should be noted that the techniques described herein may be implemented using other types of systems/devices, without limitation.

FIG. 1 illustrates various example components of the system 100. For example, FIG. 1 illustrates an implementation in which the system includes processor(s) 102, storage 104, sensor(s) 106, I/O system(s) 108, and communication system(s) 110. Although FIG. 1 illustrates a system 100 as including particular components, one will appreciate, in view of the present disclosure, that a system 100 may comprise any number of additional or alternative components.

The processor(s) 102 may comprise one or more sets of electronic circuitries that include any number of logic units, registers, and/or control units to facilitate the execution of computer-readable instructions (e.g., instructions that form a computer program). Such computer-readable instructions may be stored within storage 104. The storage 104 may comprise computer-readable recording media and may be volatile, non-volatile, or some combination thereof. Furthermore, storage 104 may comprise local storage, remote storage (e.g., accessible via communication system(s) 110 or otherwise), or some combination thereof. Additional details related to processors (e.g., processor(s) 102) and computer storage media (e.g., storage 104) will be provided hereinafter.

In some implementations, the processor(s) 102 may comprise or be configurable to execute any combination of software and/or hardware components that are operable to facilitate processing using machine learning models or other artificial intelligence-based structures/architectures. For example, processor(s) 102 may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

As will be described in more detail, the processor(s) 102 may be configured to execute instructions stored within storage 104 to perform certain actions. The actions may rely at least in part on data stored on storage 104 in a volatile or non-volatile manner. In some instances, the actions may rely at least in part on communication system(s) 110 for receiving data from remote system(s) 112, which may include, for example, separate systems or computing devices, sensors, and/or others. The communications system(s) 110 may comprise any combination of software or hardware components that are operable to facilitate communication between on-system components/devices and/or with off-system components/devices. For example, the communications system(s) 110 may comprise ports, buses, or other physical connection apparatuses for communicating with other devices/components. Additionally, or alternatively, the communications system(s) 110 may comprise systems/components operable to communicate wirelessly with external systems and/or devices through any suitable communication channel(s), such as, by way of non-limiting example, Bluetooth, ultra-wideband, WLAN, infrared communication, and/or others.

FIG. 1 illustrates that a system 100 may comprise or be in communication with sensor(s) 106. Sensor(s) 106 may comprise any device for capturing or measuring data representative of perceivable phenomena. By way of non-limiting example, the sensor(s) 106 may comprise one or more image sensors, microphones, thermometers, barometers, magnetometers, accelerometers, gyroscopes, and/or others. For example, the sensor(s) 106 may include inertial measurement unit(s) (IMU(s)), which may comprise any number of accelerometers, gyroscopes, and/or magnetometers to capture motion data associated with the system 100 as the system moves within physical space. The motion data may comprise or be used to generate pose data, which may describe the position and/or orientation (e.g., 6 degrees of freedom pose) and/or change of position (e.g., velocity and/or acceleration) and/or change of orientation (e.g., angular velocity and/or angular acceleration) of the system 100. The pose data may be used to facilitate extended reality experiences.

Furthermore, FIG. 1 illustrates that a system 100 may comprise or be in communication with I/O system(s) 108. I/O system(s) 108 may include any type of input or output device such as, by way of non-limiting example, a touch screen, a mouse, a keyboard, a controller, and/or others, without limitation. For example, the I/O system(s) 108 may include a display system that may comprise any number of display panels, optics, laser scanning display assemblies, and/or other components.

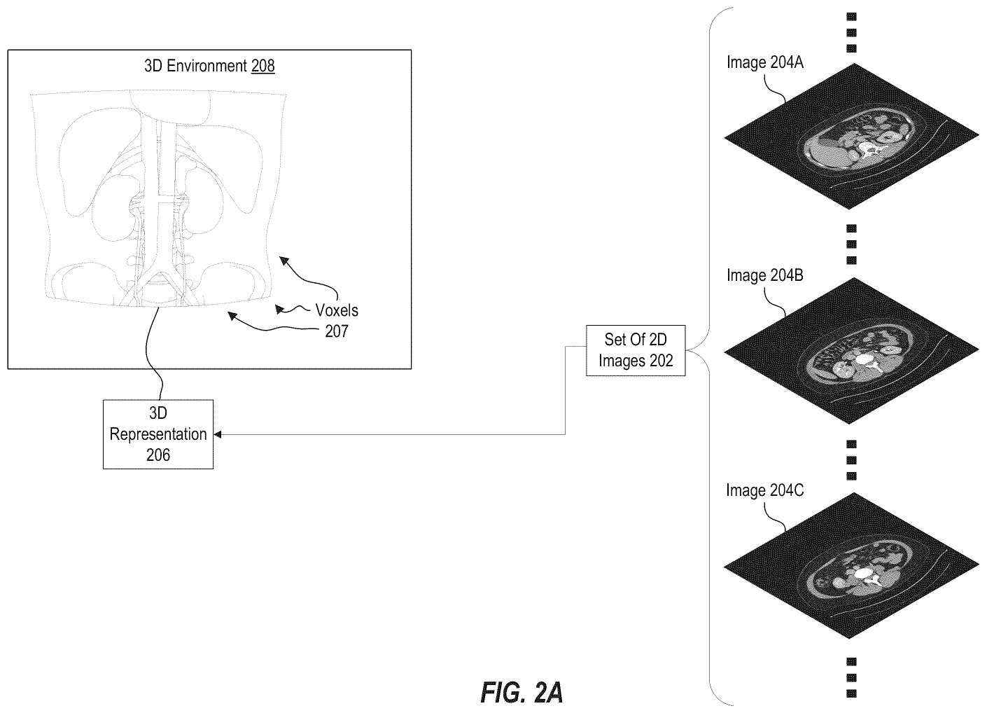

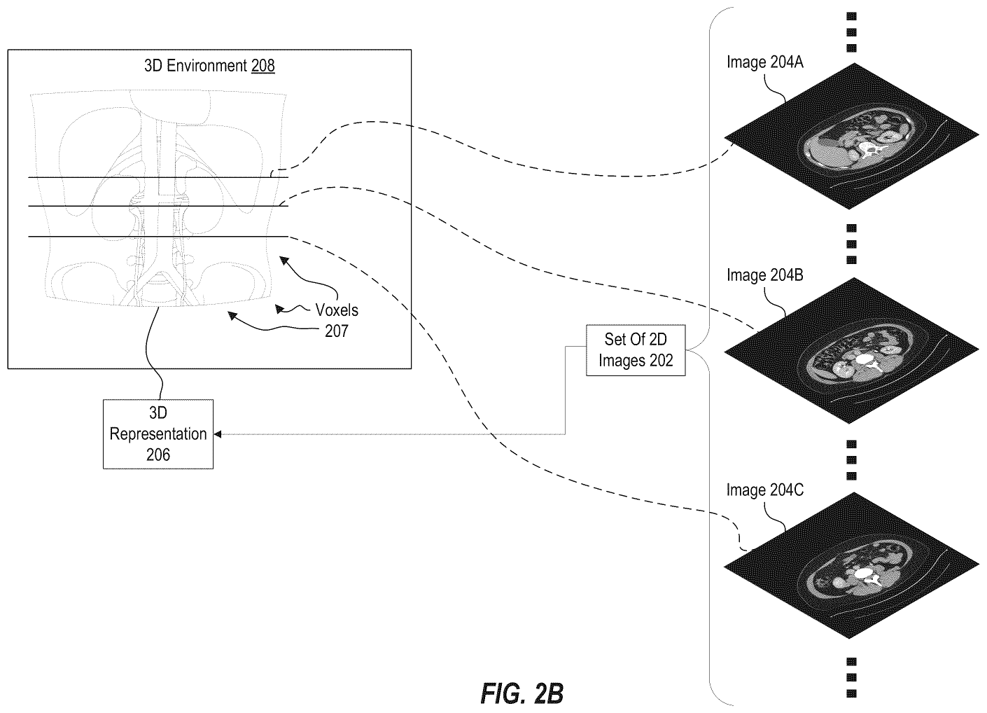

FIGS. 2A and 2B illustrate an example of a subject-specific 3D representation of anatomical structures of a subject based on a set of 2D images of the subject. In particular, FIG. 2A illustrates a set of 2D images 202 and three example images thereof (i.e., image 204A, image 204B, and image 204C). The set of 2D images 202 may comprise any number of images, as indicated by the ellipses. The set of 2D images 202 captures a particular imaging subject (e.g., a patient in a medical context). Although the example of FIG. 2A depicts the images 204A, 204B, and 204C of the set of 2D images 202 as grayscale, cross-sectional medical images (e.g., CT images of an abdomen of a human patient), the principles discussed herein may be applied utilizing any type of 2D images. For instance, a set of 2D images 202 may comprise images of any imaging modality, such as MRI images, x-ray images, ultrasound images, and/or others.

FIG. 2A furthermore shows a 3D representation 206 presented within a virtual environment 208 (e.g., a virtual 3D environment). The virtual environment 208 showing the 3D representation 206 may be presented/displayed on any suitable device, such as an extended reality head-mounted display (HMD) (e.g., system 100), a desktop computer, a tablet, a mobile electronic device, and/or others.

The subject-specific 3D representation 206 may be generated based upon the set of 2D images 202, as indicated by the arrow extending from the set of 2D images 202 to the 3D representation 206 in FIG. 2A. For example, the voxels 207 of the 3D representation 206 may be generated based on pixels of the images of the set of 2D images 202. For instance, FIG. 2B illustrates dashed lines extending from the images 204A, 204B, and 204C of the set of 2D images 202 toward different cross-sectional portions of the 3D representation 206, indicating that the voxels 207 forming the cross-sectional portions of the 3D representation 206 may be generated based upon the pixel values of image pixels of the 2D images 204A, 204B, and 204C. The 3D representation 206 may thus depict the various structures (e.g., anatomical structures) represented in the set of 2D images 202 (e.g., the CT image set of the abdomen of the human patient), as shown in FIG. 2B. One will appreciate that interpolation and/or other processes may be performed to generate the voxels 207 based on the pixels of the set of 2D images 202. Furthermore, although the 3D representation 206 is generated based upon the set of 2D images 202, it will be appreciated that the 3D representation 206 need not be generated directly from the raw images of the set of 2D images 202. For example, image data of a set of raw 2D images may be loaded into one or more channels of a multi-channel texture (e.g., a 4-channel red, green, blue, alpha (RGBA) texture), while other channels of the multi-channel texture may be utilized to store other information (e.g., pixel selection information of a selection mask).

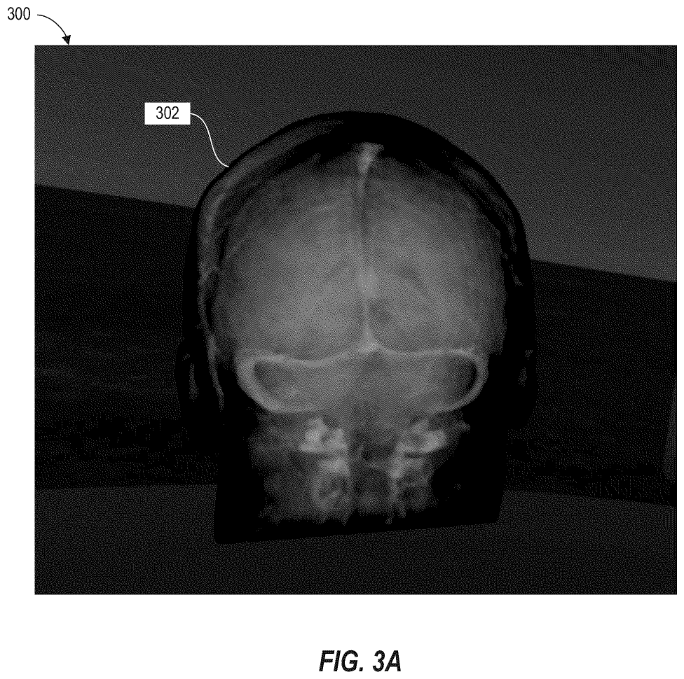

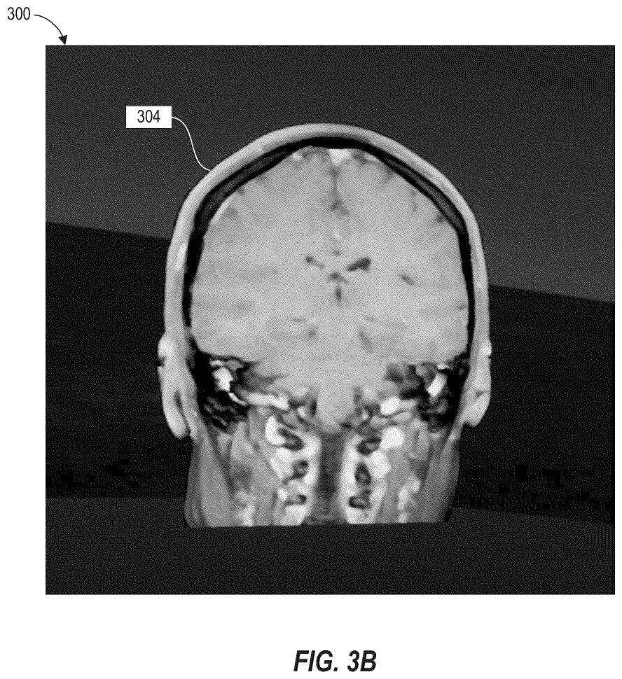

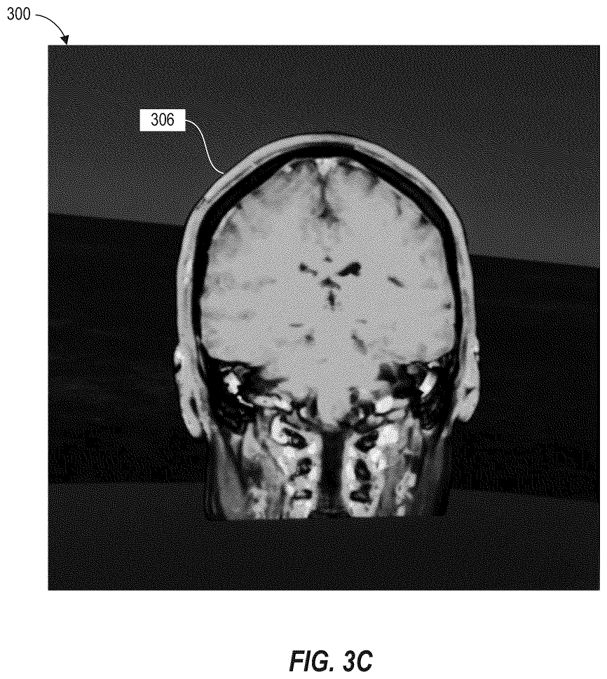

FIG. 3A provides an example of a subject-specific 3D representation 302, which may be generated in the manner described hereinabove with reference to the subject-specific 3D representation 206 of FIGS. 2A and 2B. In the example shown in FIG. 3A, the subject-specific 3D representation 302 is a 3D construction generated using 2D medical images (e.g., x-ray or CT images) of a at least part of a human subject (e.g., a human head, in the example shown in FIG. 3A). The subject-specific 3D representation 302 can be displayed to users within a virtual environment 300 in navigable form, enabling users to view and manipulate the 3D representation for educational, patient care, and/or other purposes.

FIG. 3B illustrates an alternative subject-specific 3D representation 304 that provides a grayscale representation of the same bodily structures (i.e., the head) depicted by the subject-specific 3D representation 302 of FIG. 3A. For instance, whereas the voxels of the subject-specific 3D representation 302 of FIG. 3A may have brightness, intensity, or other values defining visual characteristics taken directly from corresponding pixels of the 2D medical imagery used to generate the subject-specific 3D representation 302, the voxels of the subject-specific 3D representation 304 of FIG. 3B may be determined by applying pixel mappings or other transformations to the pixel values of the underlying 2D medical imagery (or by applying voxel mappings to voxel values of a base subject-specific 3D representation). In the example shown in FIG. 3B, the voxels of the subject-specific 3D representation 304 may be obtained by applying a mapping function to the pixel values of the underlying 2D medical imagery of the subject to achieve a higher-brightness representation of the brain of the subject. Other transformations or mappings may be performed on 2D image data representing a subject to obtain subject-specific 3D representations with different visual characteristics, such as by mapping 2D pixel values to different color values, tissue density values, transparency values, and/or other values that may be represented in voxels for displaying a subject-specific 3D representation. For example, FIG. 3C illustrates a subject-specific 3D representation 306 generated by applying a color mapping to pixel values of the 2D image data capturing the subject, thereby providing a colorized representation.

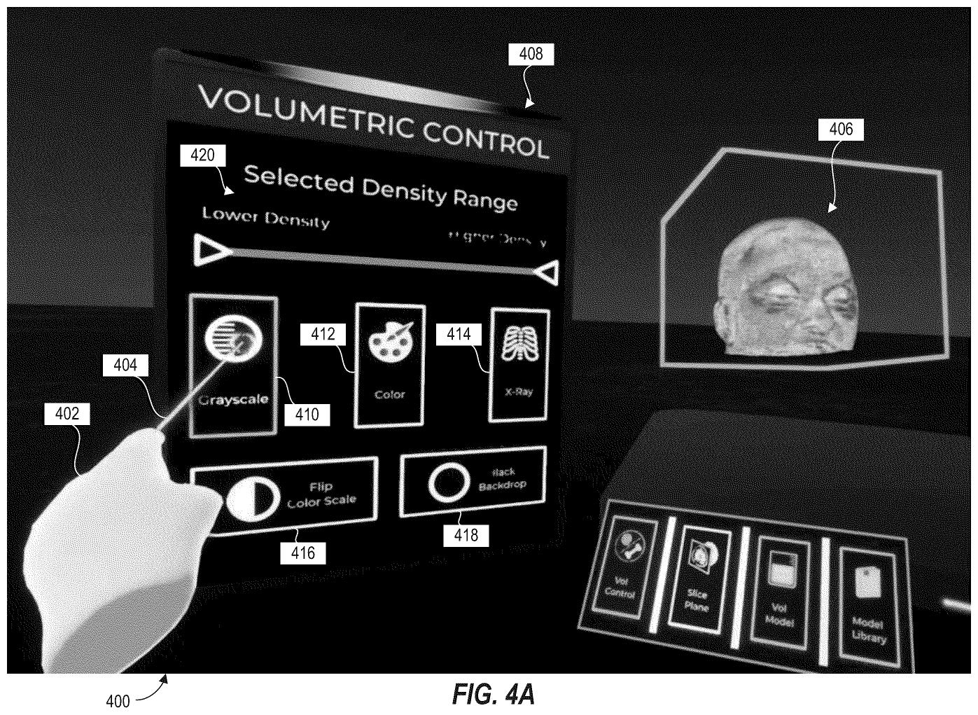

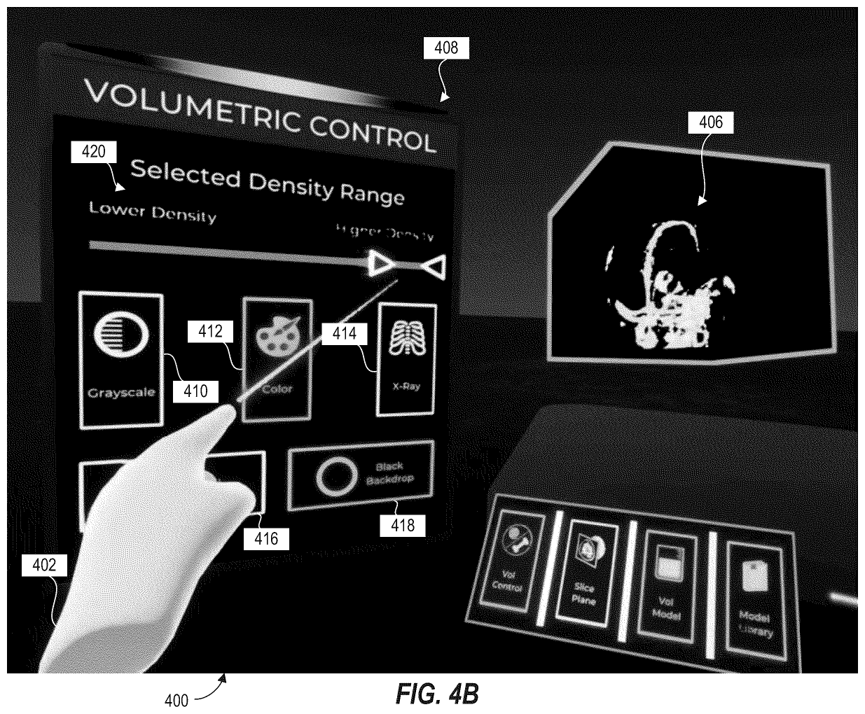

FIGS. 4A and 4B illustrate an example user interface for receiving input directed to changing presentation characteristics of a subject-specific 3D representation of an anatomical structure of a subject. FIG. 4A depicts a virtual environment 400 that may be presented on one or more user devices (e.g., a system 100) to enable users to perceive and/or interact with content within the virtual environment 400. For example, the virtual environment 400 can comprise a virtual reality environment or augmented reality environment that the user accesses via an extended reality device (e.g., a head-mounted display). A user operating the extended reality device can navigate through the virtual environment 400 via various control actions (e.g., head movements, locomoting, hand movements and/or gestures, manipulation of one or more controllers, etc.). In the example shown in FIG. 4A, the virtual environment 400 depicts a control visualization 402 embodied as a representation of a user's hand. The control visualization 402 may be presented based on sensor data that captures real-world movements of the user's hands, providing an immersive experience for the user. The virtual environment 400 also depicts a control ray 404 extending from the control visualization 402, which can enable users to finely select or control virtual objects and/or interactable elements within the virtual environment 400.

Although the example(s) described with reference to FIG. 4A (and other Figures herein) focus, in at least some respects, on extended reality implementations, the principles disclosed herein can be implemented using other types of virtual environments (e.g., 3D environments accessible via a desktop or laptop computer or other device such as a smartphone, tablet, etc.).

FIG. 4A shows an example in which a subject-specific 3D representation 406 is presented within the virtual environment 400. The user may navigate through the virtual environment 400 to perceive and/or interact with the subject-specific 3D representation 406 from multiple perspectives. FIG. 4A also illustrates a control panel 408 that can provide users with various functions for changing presentation characteristics for the subject-specific 3D representation 406. For instance, the control panel 408 includes selectable elements 410, 412, and 414 that can be selected to cause the subject-specific 3D representation 406 to embody different visual characteristics. For instance, selectable element 410 can be selected to cause the subject-specific 3D representation 406 to appear as a grayscale model (similar to subject-specific 3D representation 304), selectable element 412 can be selected to cause the subject-specific 3D representation 406 to appear as a color model (similar to subject-specific 3D representation 306), and selectable element 414 can be selected to cause the subject-specific 3D representation 406 to appear as an x-ray or CT image model (similar to subject-specific 3D representation 302). Users can thus readily modify visual characteristics of the subject-specific 3D representation 406 for different use contexts.

The example control panel 408 shown in FIG. 4A also includes other features, such as selectable element 416 (e.g., which can cause changing of the color scale for presenting the subject-specific 3D representation 406 within the virtual environment 400) and selectable element 418 (e.g., which can toggle whether a black backdrop is included when presenting the subject-specific 3D representation 406 within the virtual environment 400). Furthermore, the example control panel 408 is depicted as including a display filter 420 implemented as a sliding scale that users may manipulate to influence a degree of voxel filtering for display of the subject-specific 3D representation 406. As indicated above, voxels of the subject-specific 3D representation 406 may be associated with different tissue density values (e.g., based on intensity or other pixel values from 2D imagery), and the display filter 420 may determine one or more tissue density thresholds or ranges for display of the subject-specific 3D representation 406, such that voxels associated with tissue density values that fall outside of the tissue density threshold(s) or range(s) are hidden (or made transparent). Such functionality may enable users to incrementally hide or make transparent softer tissues to expose denser tissues, or vice-versa, which may provide valuable educational experiences for users. Tissue density may similarly operate as a constraint for display of an idealized 3D representation, where tissue density labels may be associated with different surfaces/meshes of the idealized 3D representation.

FIG. 4B depicts presentation of the subject-specific 3D representation 406 within the virtual environment 400 as a color model (e.g., based on selection of selectable element 412) with a black backdrop (e.g., based on the toggle state of selectable element 418) with emphasis on certain voxels that fall within a reduced tissue density range (e.g., defined via user input directed to display filter 420).

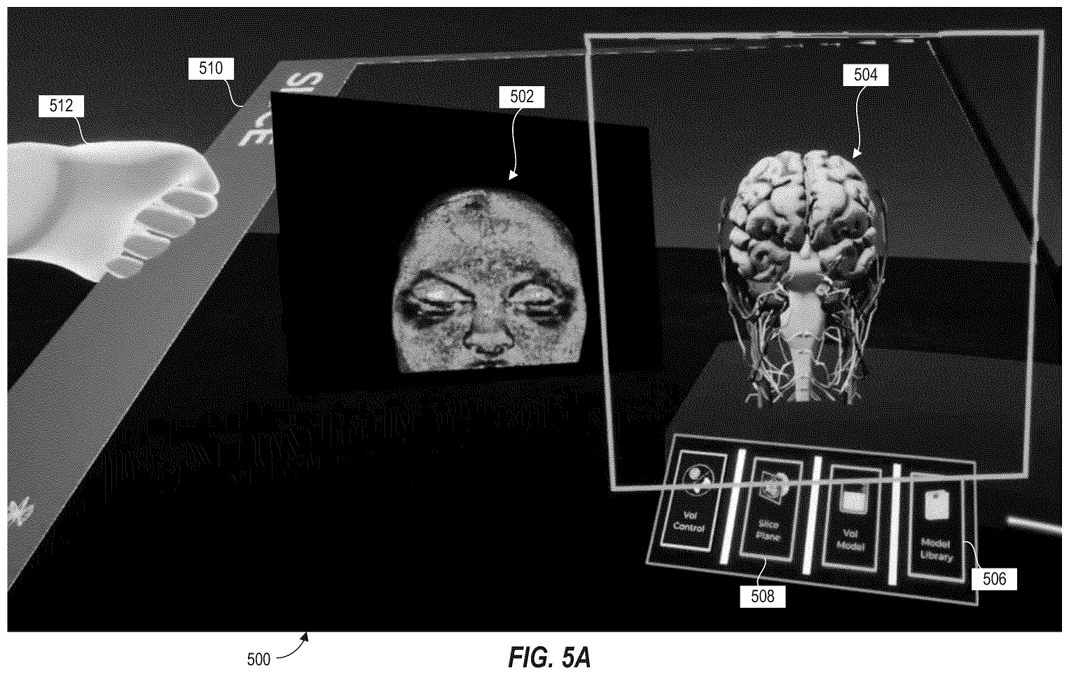

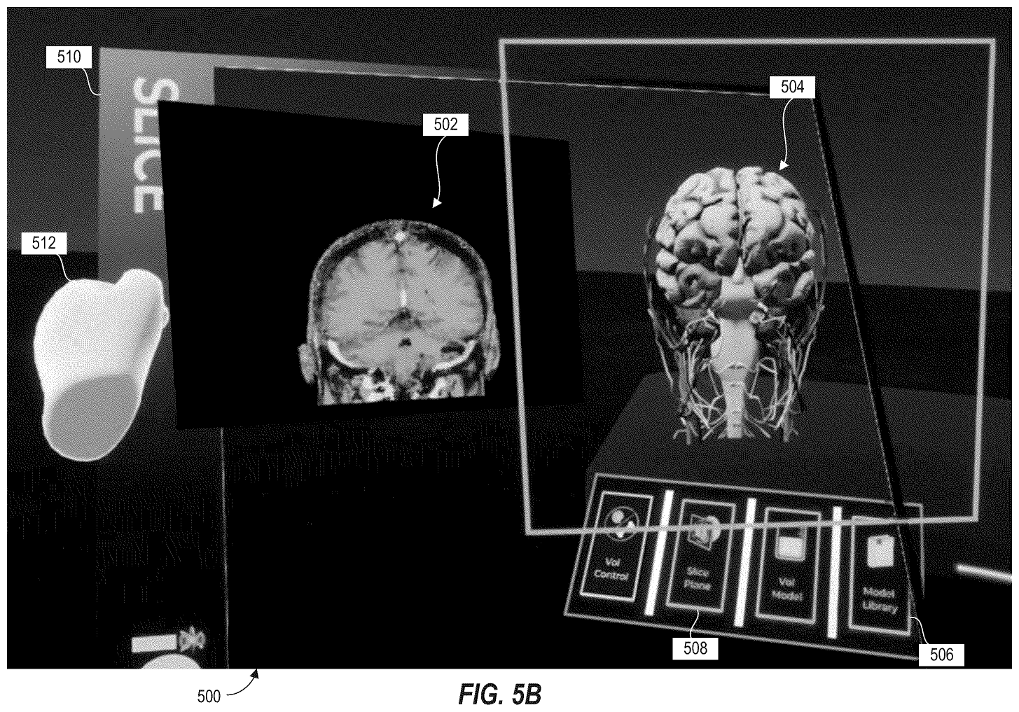

FIGS. 5A and 5B illustrate an example virtual environment 500 in which a subject-specific 3D representation 502 of an anatomical structure (e.g., a subject's head) is presented for perception and/or interaction by a user. The subject-specific 3D representation 502 may be generated based on a set of 2D images, as described hereinabove. In the example shown in FIG. 5A, an idealized 3D representation 504 of the same anatomical structure (e.g., a head) is simultaneously presented within the virtual environment 500, enabling the user to navigate among and/or interact with both 3D representations. As used herein, simultaneous display of both a subject-specific 3D representation and an idealized 3D representation within a virtual environment refers to a state in which both the subject-specific 3D representation and the idealized 3D representation are virtually positioned in a view frustum within the virtual environment.

In the example shown in FIG. 5A, the idealized 3D representation 504 is not generated/constructed using, as processing inputs, 2D cross-sectional image data of a real patient (e.g., the idealized 3D representation 504 is instead manually designed and/or computer generated). The idealized 3D representation 504 may be user-selected for display in conjunction with the subject-specific 3D representation 502 from a library or plurality of idealized 3D models/representations based on various factors. The virtual environment 500 depicts a selectable element 506 that a user may select to surface a model library from which to select an idealized 3D representation to present in conjunction or simultaneity with the subject-specific 3D representation 502.

In some implementations, the idealized 3D representation 504 is dynamically or automatically selected (e.g., in response to an initial user command to instantiate an idealized 3D representation within the virtual environment 500). For example, the idealized 3D representation 504 may be selected based on one or more attributes of the subject represented in the subject-specific 3D representation 502, such as age, gender, ethnic background, height, weight, pathologies experienced/exhibited, and/or other factors. In some instances, the idealized 3D representation 504 is selected based on a use context, such as an educational purpose (e.g., based on instruction or testing/assessment context, based on the pathologies that are the subject of instruction or assessment, etc.), clinical context, etc. In some instances, the idealized 3D representation 504 displayed in conjunction with the subject-specific 3D representation 502 is a modified version of a base idealized model selected from a database of idealized models (e.g., from the model library accessible via selectable element 506). For instance, cosmetic changes, segmenting/sectioning, filtering, and/or other transformations may be applied to a base idealized model to obtain the idealized 3D representation 504.

The idealized 3D representation 504 can thus be selected (manually or automatically) or generated so as to depict at least some of the same anatomical structures as the subject-specific 3D representation 502. Simultaneous display of both the subject-specific 3D representation 502 and the idealized 3D representation 504 within the same environment can be utilized as an educational tool to help users achieve an intuitive understanding of human anatomical structures, as well as how they can deviate from patient to patient. Although a head is shown in FIG. 5A, other anatomical structures may be represented by a simultaneously displayed subject-specific and idealized 3D representation, in accordance with the disclosed principles.

A user may utilize various virtual tools to interact with or otherwise examine a subject-specific and/or idealized 3D representation within a virtual environment. For instance, FIG. 5A depicts a selectable element 508 that may be selected to activate a slice plane 510, which the user may manipulate to generate sliced or sectioned visualizations of the subject-specific 3D representation 502. In the example shown in FIG. 5A, the slice plane 510 has been activated by selection of selectable element 508, and the slice plane 510 is selectively tethered to the control visualization 512, enabling the user to manipulate the positioning of the slice plane 510 by moving the control visualization 512 (e.g., which can be accomplished by moving the user's hand or another real-world controller).

Under the positioning shown in FIG. 5A, the slice plane 510 does not, within the 3D space of the virtual environment 500, intersect with the volume of the subject-specific 3D representation 502. FIG. 5B illustrates an instance in which the slice plane 510 is brought into intersection with the volume of the subject-specific 3D representation 502, thereby providing a sectioned or sliced representation of the subject-specific 3D representation 502 where voxels of the subject-specific 3D representation 502 that are between the slice plane 510 and the user's viewing position are made transparent or are otherwise visually modified (e.g., to allow interior features of the subject-specific 3D representation 502 to become visible to the user). The sectioned or sliced representation of the subject-specific 3D representation 502 can be automatically updated in near-real-time based on movements of the slice plane 510. The sectioned or sliced representation of the subject-specific 3D representation 502 can thus allow users to examine interior features of the subject-specific 3D representation 502 in an intuitive manner, which can provide users with useful insights.

Additional example virtual tools for interacting with or otherwise examining a subject-specific and/or idealized 3D representation within a virtual environment will be described hereinafter.

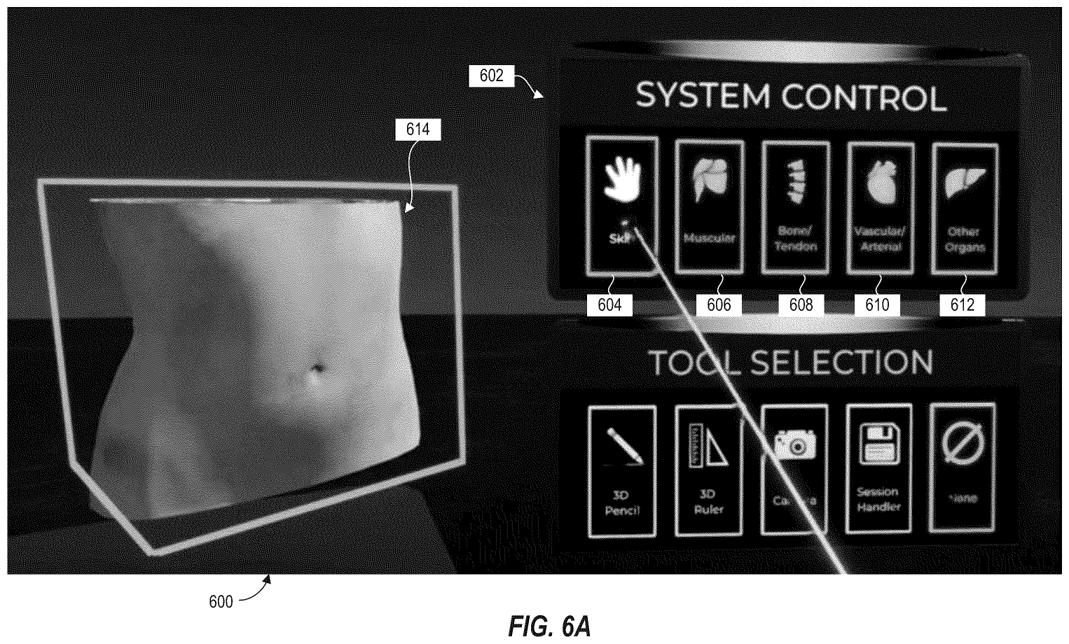

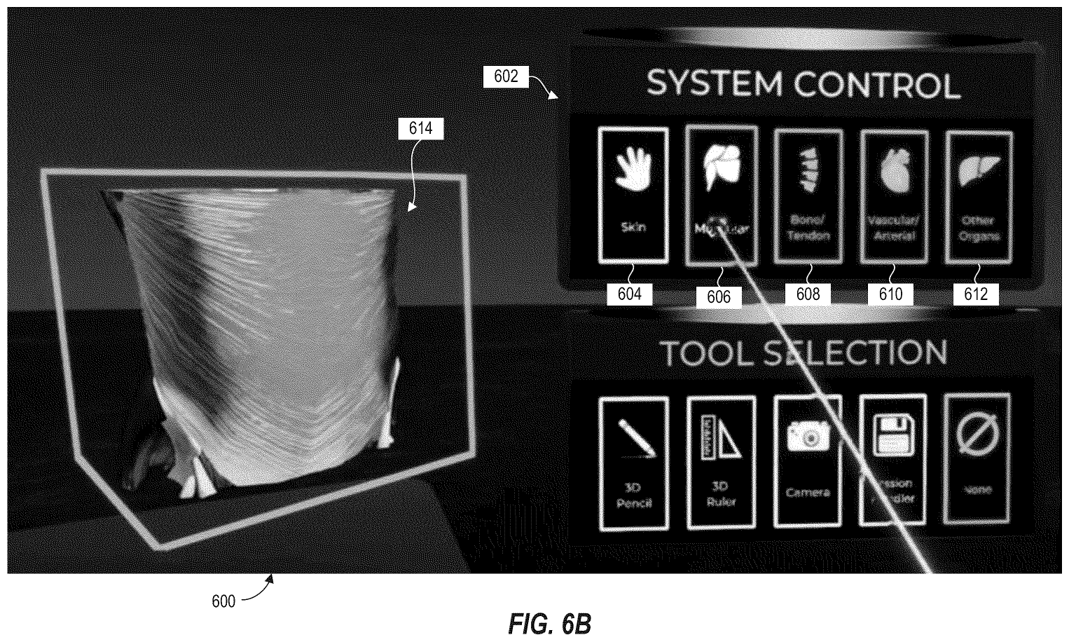

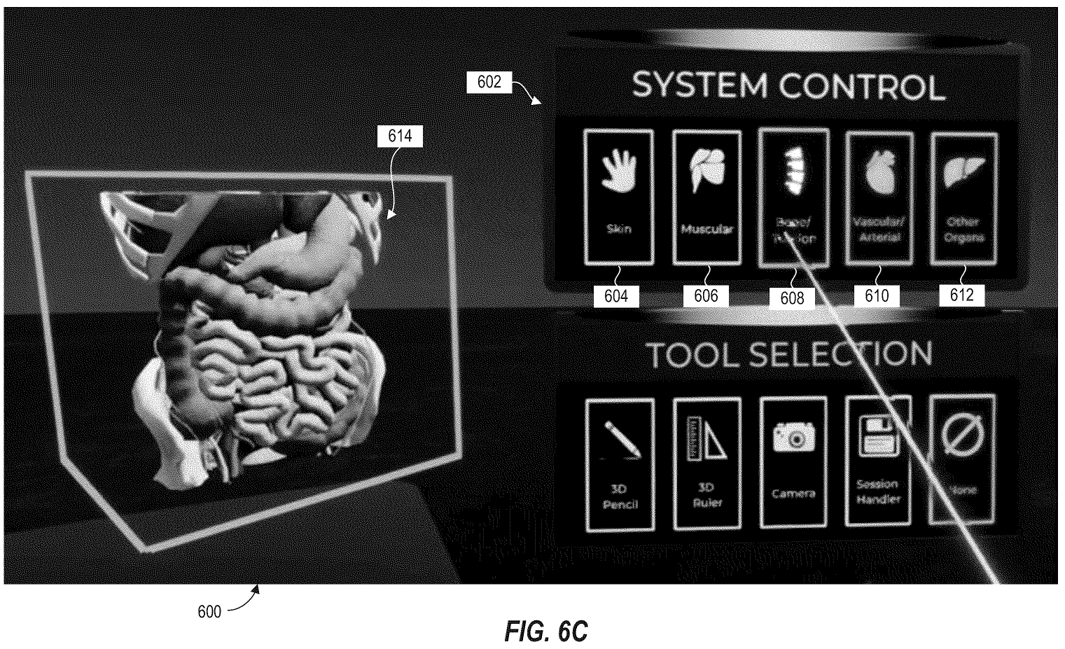

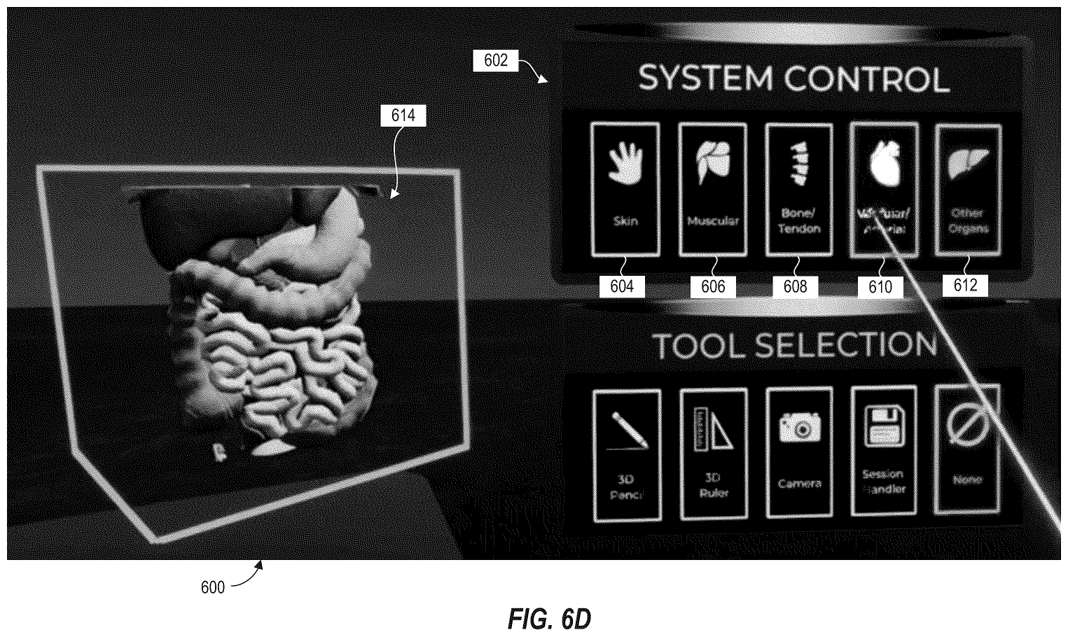

In some instances, an idealized 3D representation presented within a virtual environment depicts (or is modified to depict) an isolated anatomical system, such as an integumentary, muscular, skeletal, circulatory, lymphatic, respiratory, digestive, nerve, or other bodily system. FIGS. 6A, 6B, 6C, and 6D illustrate example idealized 3D representations of different anatomical systems/structures. FIG. 6A illustrates a virtual environment 600 that includes a control panel 602 that includes selectable elements 604, 606, 608, 610, and 612 that can allow users to activate or deactivate presentation layers associated with an idealized 3D representation 614. Each of the different presentation layers is associated with a different bodily system. For instance, in FIG. 6A, all presentation layers are active (e.g., selectable elements 604, 606, 608, 610, and 612 are all in an active or “on” state), causing the idealized 3D representation 614 to include skin features (associated with selectable element 604), muscular features (associated with selectable element 606), bone/tendon features (associated with selectable element 608), vascular/arterial features (associated with selectable element 610), and other organ features (associated with selectable element 612). In FIG. 6B, the skin layer is inactive (with selectable element 604 in an inactive or “off” state), causing the idealized 3D representation 614 to omit skin features. In FIG. 6C, both the skin layer and the muscular layer are inactive (with selectable elements 604 and 606 in the inactive or “off” state), causing the idealized 3D representation 614 to omit skin and muscular features. In FIG. 6D, the skin layer, the muscular layer, and the bone/tendon layer are inactive (with selectable elements 604, 606, and 608 in the inactive or “off” state), causing the idealized 3D representation 614 to omit skin, muscular, and bone/tendon features.

Other layer definitions and/or layouts for selecting which bodily features/structures to include in an idealized 3D representation are within the scope of the present disclosure, and different layer definitions and/or layouts may be used for different use cases.

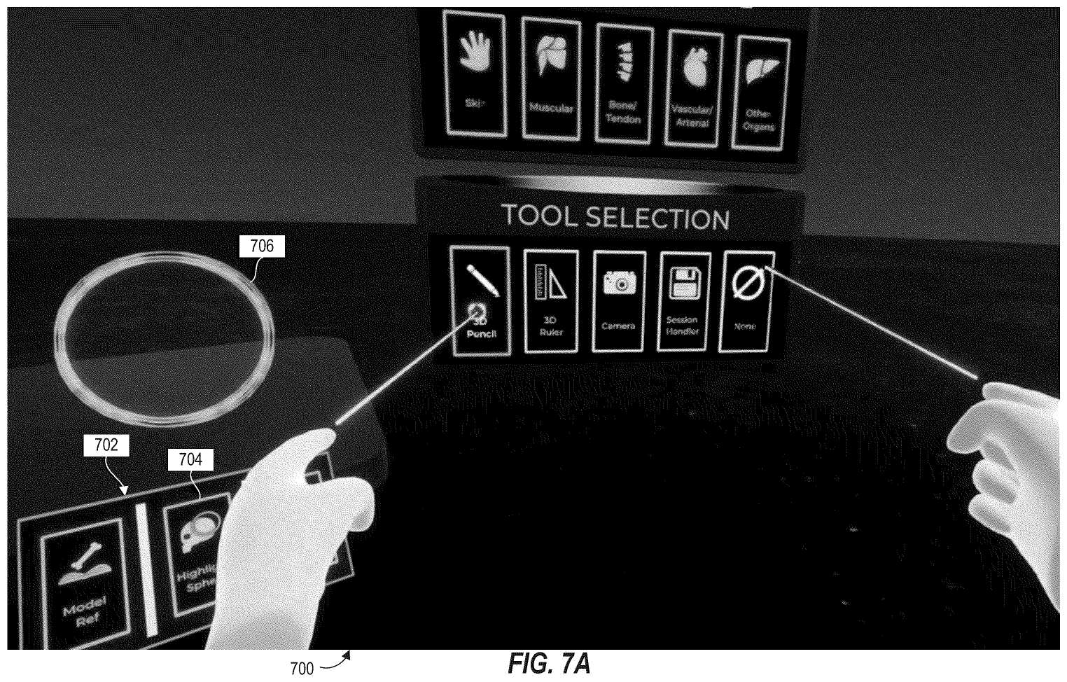

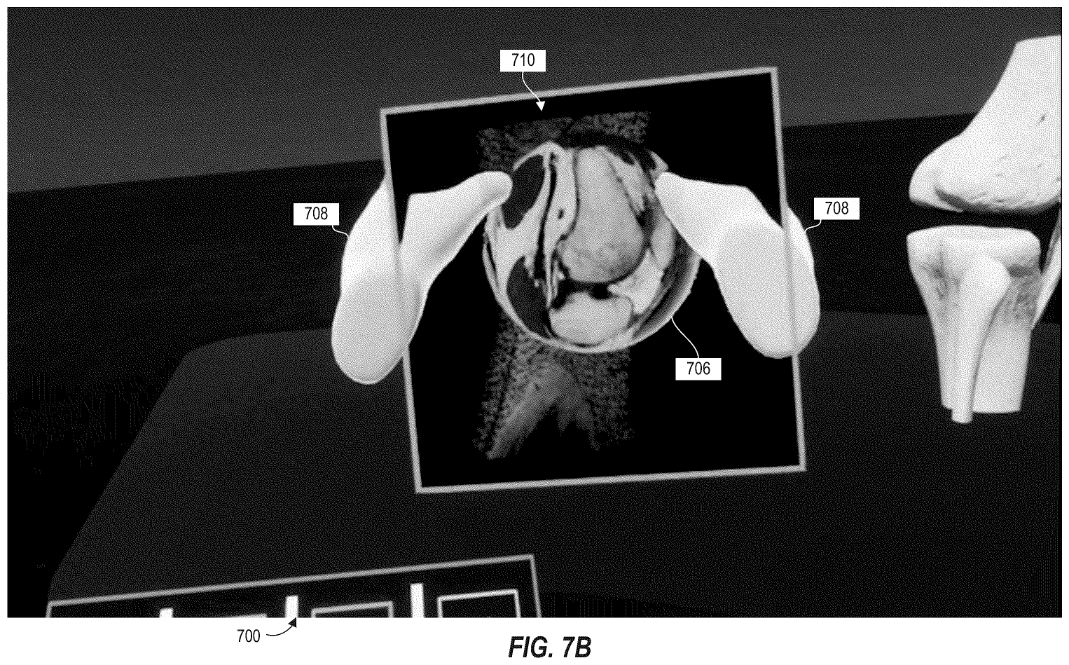

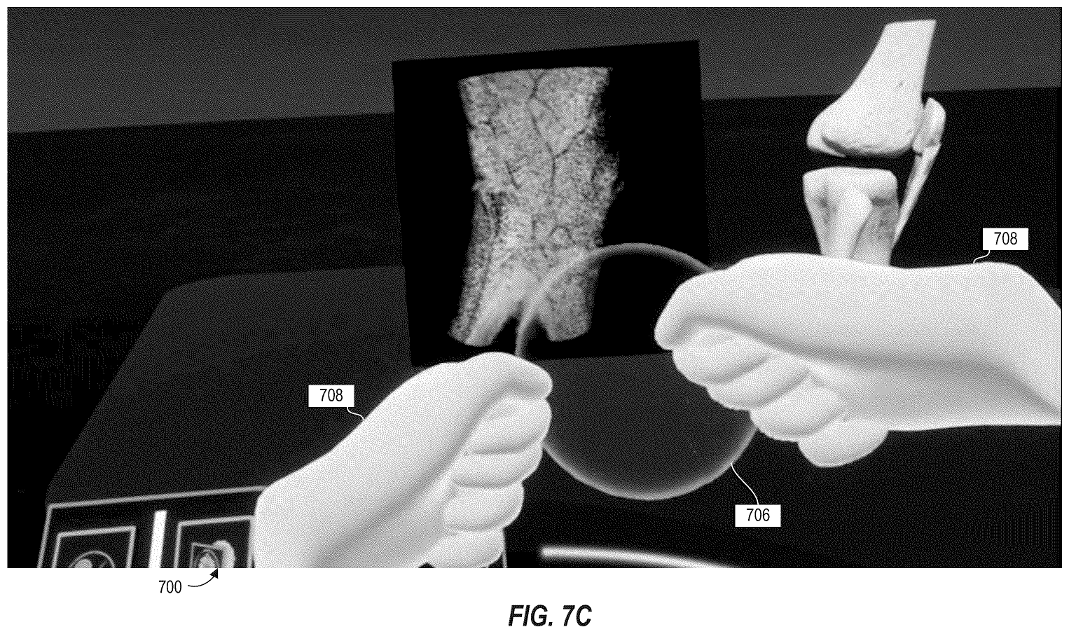

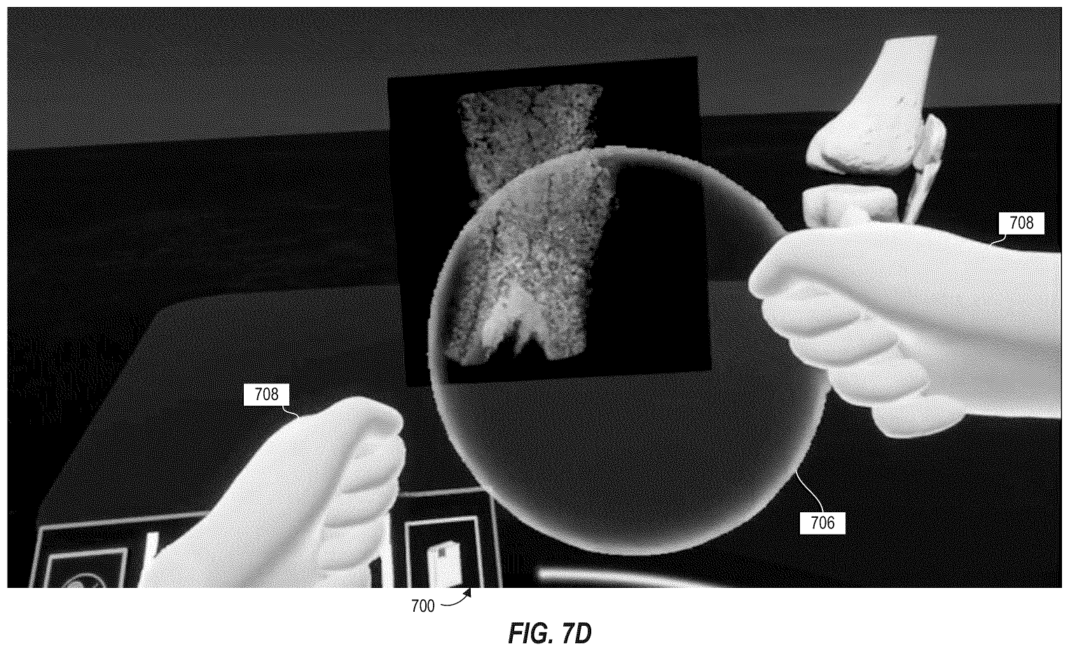

FIG. 7A illustrates a virtual environment 700 in which subject-specific and/or idealized 3D representations may be presented to users. The virtual environment 700 depicts control panel 702 that includes selectable elements associated with various virtual tools that can be used to interact with or otherwise examine subject-specific and/or idealized 3D representations. For instance, the control panels 702 is depicted in FIG. 7A as including selectable element 704 associated with a highlight sphere tool. In the example shown in FIG. 7A, selectable element 704 has been selected, triggering instantiation of a highlight sphere 706 within the virtual environment 700. The highlight sphere 706 can operate in a manner conceptually similar to the slice plane 510 discussed hereinabove, in that the positioning of the highlight sphere 706 can be manipulated via user input, and voxels contained by the highlight sphere 706 can be made transparent or otherwise visually modified (e.g., to allow interior features of subject-specific and/or idealized 3D representations to become visible to users).

FIG. 7B illustrates an instance in which the highlight sphere 706 is moved within the virtual environment 700 in connection with the control visualizations 708 (controlled by the user's real-world movements/operations) to a position where the highlight sphere 706 contains certain voxels of a subject-specific 3D representation 710, causing the contained voxels to become hidden/removed or to appear transparent. Such functionality can allow users to view internal features of the subject-specific 3D representation 710 in an intuitive manner.

In some implementations, the size, shape, and/or other characteristics of a highlight sphere tool may be modified by users. By way of illustrative example, FIGS. 7C and 7D conceptually depict user input directed to the highlight sphere 706 via the control visualizations 708 in the form of pinching or gripping gestures directed toward the highlight sphere 706 (shown in FIG. 7C) and subsequent repositioning of the control visualizations 708 (drawing the control visualizations 708 away from one another, as shown in FIG. 7D) while maintaining the pinching or gripping gestures to cause resizing of the highlight sphere 706. Other modes of resizing (e.g., selecting from available size options) or types of voxel modifications (e.g., controlling which types of tissue are modified when contained by the highlight sphere 706) associated with a highlight sphere may be implemented in other examples.

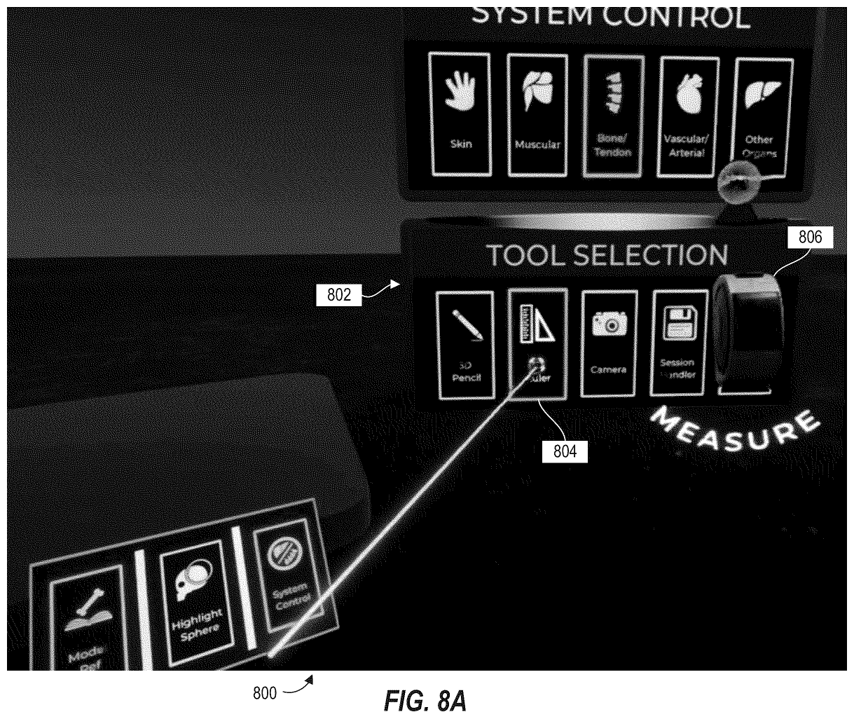

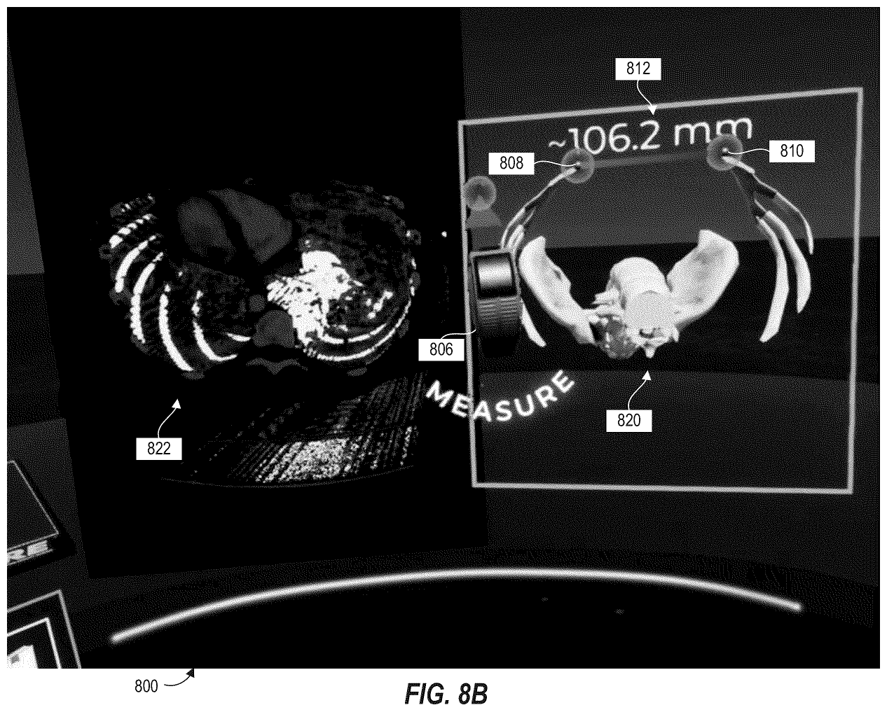

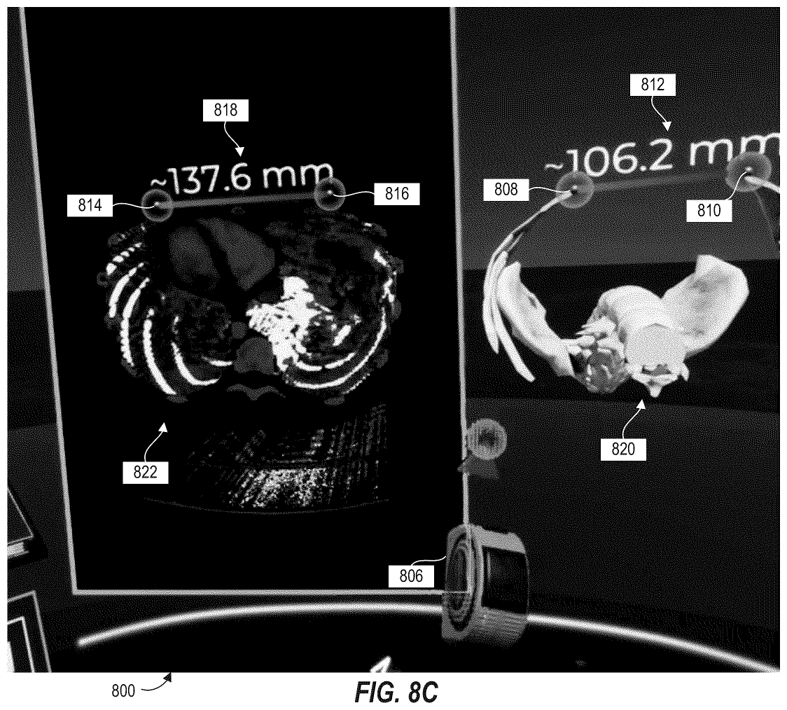

FIG. 8A illustrates another virtual environment 800 in which subject-specific and/or idealized 3D representations may be presented to users. The virtual environment 800 depicts control panel 802 that includes selectable element 804 associated with a virtual measurement tool. In the example shown in FIG. 8A, selectable element 804 has been selected, triggering instantiation of a measurement tool 806 within the virtual environment 800. The measurement tool 806 can be used to measure distances between points in the virtual space of the virtual environment 800. The measurement distances can be correlated to real-world distances (e.g., in view of each voxel of a subject-specific 3D representations corresponding to a real-world volumetric size), which can enable users to make useful comparisons and/or draw useful inferences while examining subject-specific and/or idealized 3D representations.

FIG. 8B illustrates an instance in which, with the measurement tool 806 active, the user has selected two points 808 and 810 in the 3D space of the virtual environment 800 (e.g., by controlling motion of the measurement tool 806 via real-world movements/operations) to determine the distance 812 therebetween. The distance 812 between the points 808 and 810 is displayed to the user within the virtual environment 800. FIG. 8C illustrates another instance in which the measurement tool 806 is used to determine the distance 818 between two other points 814 and 816. In the example shown, points 808 and 810 are associated with a displayed idealized 3D representation 820, whereas points 814 and 816 are associated with a displayed subject-specific 3D representation 822. Such functionality can allow users to compare distances associated with subject-specific 3D representations to distances associated with idealized 3D representations, which can be useful for educational, diagnostic, or other purposes.

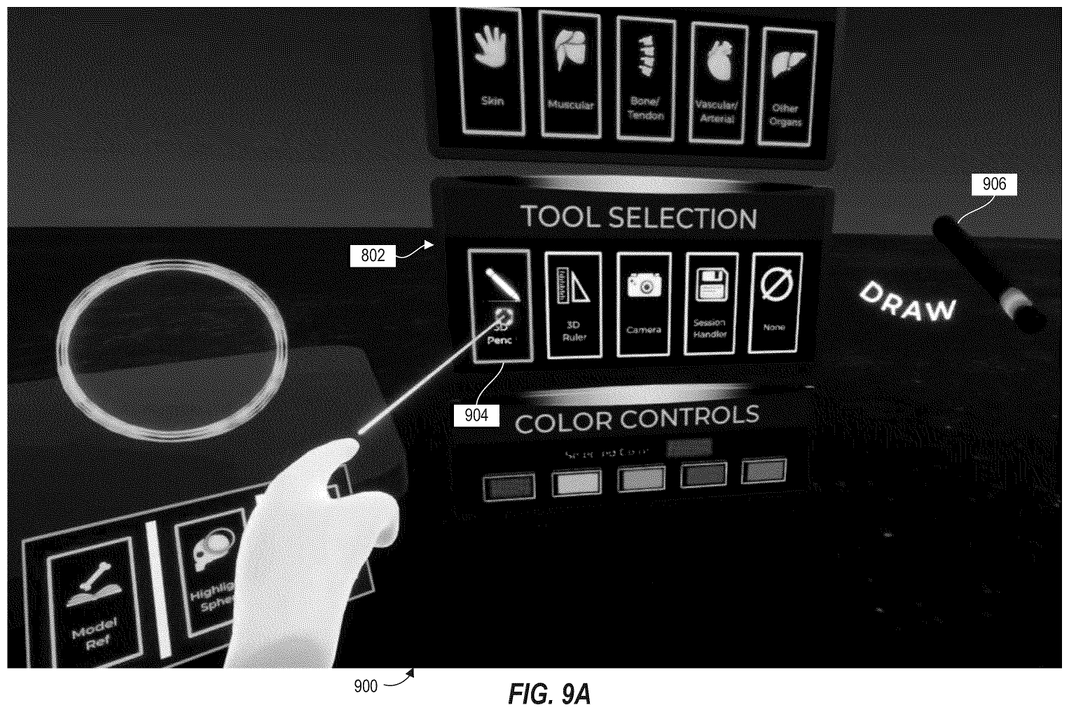

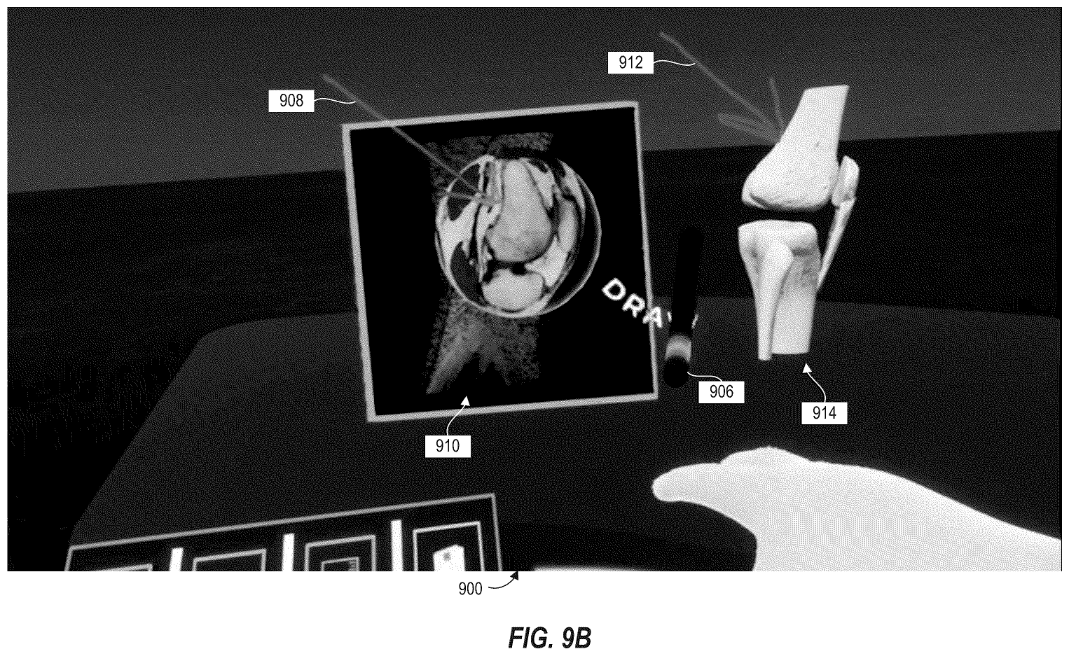

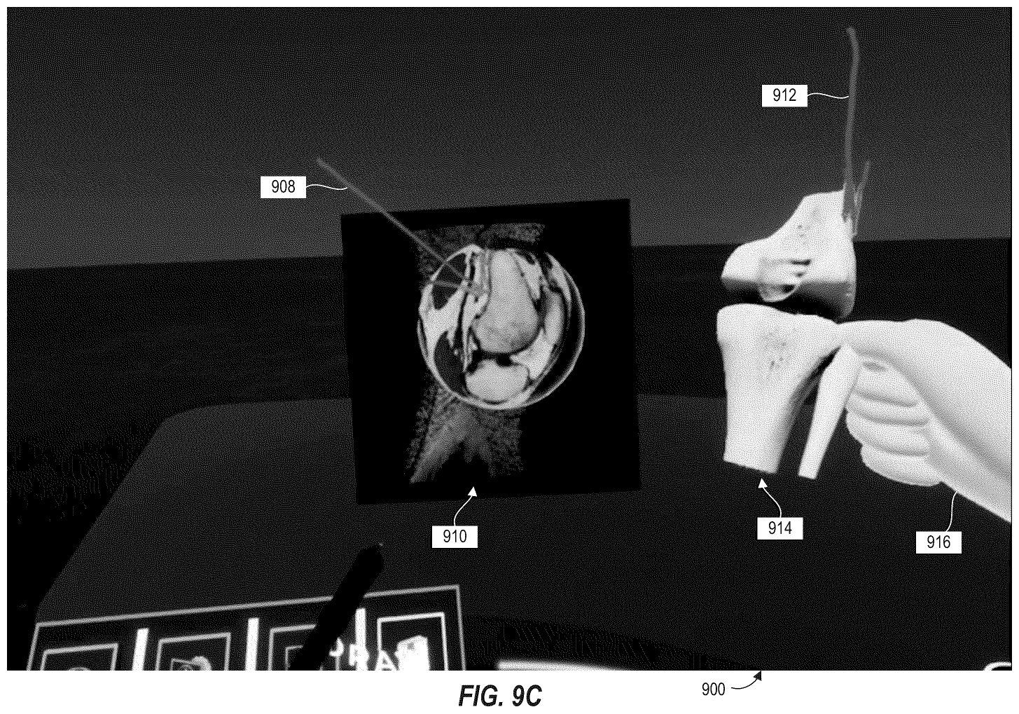

FIG. 9A illustrates another virtual environment 900 in which subject-specific and/or idealized 3D representations may be presented to users. The virtual environment 900 depicts the control panel 802 of FIG. 8A as including selectable element 904 associated with a virtual annotation tool. In the example shown in FIG. 9A, selectable element 904 has been selected, triggering instantiation of an annotation tool 906 within the virtual environment 900. The annotation tool 906 can be used to annotate a subject-specific and/or idealized 3D representation displayed within the virtual environment 900, which can be useful for educational, assessment, diagnostic, and/or other purposes.

FIG. 9B illustrates an instance in which the annotation tool 906 has been used (e.g., by controlling motion of the annotation tool 906 via real-world movements/operations) to draw an annotation 908 in association with a subject-specific 3D representation 910 and an annotation 912 in association with an idealized 3D representation 914 within the virtual environment 900. In the example shown in FIG. 9B, the annotations 908 and 912 comprise hand-drawn arrows directed toward human anatomical features that are present in both the subject-specific 3D representation 910 and the idealized 3D representation 914. Such functionality can enable users to readily learn associations between anatomical components and how they may be embodied in particular patients/subjects.

One will appreciate, in view of the present disclosure, that annotations associated with subject-specific and/or idealized 3D representations can take on various forms, such as highlighting, text labeling, and/or others. Although FIG. 9B focuses on an example in which the annotations 908 and 912 are hand-drawn, annotations may be generated in association with an idealized and/or subject-specific 3D representation in other ways, such as by the user selecting a component within one or both of the subject-specific and/or the idealized 3D representation(s), or by the user selecting an anatomical label or name from a list or other data structure. In some instances, annotations are generated and displayed in association with an idealized and/or subject-specific 3D representation after the occurrence of other events, such as while the user proceeds through an instructional workflow within a virtual environment.

In some instances, the portions, components, or aspects of the anatomical structures of a subject-specific 3D representation and/or an idealized 3D representation that become visually emphasized with annotations are associated with patient care actions. Such functionality can enable users to prepare to accurately perform patient care actions with real patients. By way of illustrative example, portions of a subject-specific 3D representation and/or an idealized 3D representation that become visually emphasized may correspond to locations on a subject's body where a medical practitioner should feel to detect the subject's pulse or locations on a subject's abdomen where a medical practitioner should utilize a stethoscope to assess heart or lung functioning.

In some implementations, the annotations or labels that become displayed in association with an idealized and/or subject-specific 3D representation may be determined based on information obtained from a database (e.g., metadata or other data associated with an idealized 3D representation) or based on one or more pre-processing operations (performed on data that forms a basis of a subject-specific 3D representation, such as a set of 2D images). For example, a subject-specific 3D representation and/or the set of 2D images associated therewith may be pre-processed utilizing one or more machine learning modules that determine anatomical labels for detected structures. Such anatomical labels may be utilized to annotate the subject-specific 3D representation during display thereof within a virtual environment (e.g., in conjunction with an idealized 3D representation).

In some instances, users are prompted to label or annotate anatomical structures represented in one or both of the subject-specific and/or the idealized 3D representation (e.g., in a testing or assessment context). Such annotations may take on various forms, such as text, measurements, drawings, and/or others for various purposes (e.g., personal note-taking, testing, etc.).

A subject-specific 3D representation and/or an idealized 3D representation may be presented in a navigable or modifiable form within a virtual environment, allowing users to modify and/or manipulate positioning and/or other presentation characteristics of the subject-specific and/or the idealized 3D representation. Such functionality may allow users to achieve interaction with and/or immersive examination of the displayed 3D representations, which can improve educational outcomes.

For instance, FIG. 9C depicts the subject-specific 3D representation 910 and the idealized 3D representation 914 within the virtual environment 900 discussed above. FIG. 9C also conceptually depicts a control visualization 916 that is controllable by user actions to enable the user to provide user input to influence presentation characteristics for presenting the subject-specific 3D representation 910 and/or the idealized 3D representation 914. Responsive to such input, a system may modify presentation of the subject-specific 3D representation 910 and/or the idealized 3D representation 914. In the example shown in FIG. 9C, the control visualization 916 is engaged with the idealized 3D representation 914 (e.g., indicated by a pinching or grabbing gesture embodied by the control visualization 916), allowing subsequent repositioning of the control visualization 916 to cause modification of the positioning of the idealized 3D representation 914. FIG. 9C illustrates an instance where such positional modifications have occurred, showing the idealized 3D representation 914 at a different position and orientation than those shown in FIG. 9B.

In some implementations, annotations may maintain their positions relative to their associated idealized and/or subject-specific 3D representation throughout positional modifications (and/or other modifications), as shown in FIG. 9C, which illustrates annotation 912 maintaining its position relative to the idealized 3D representation 914 throughout the change in the position and orientation of the idealized 3D representation.

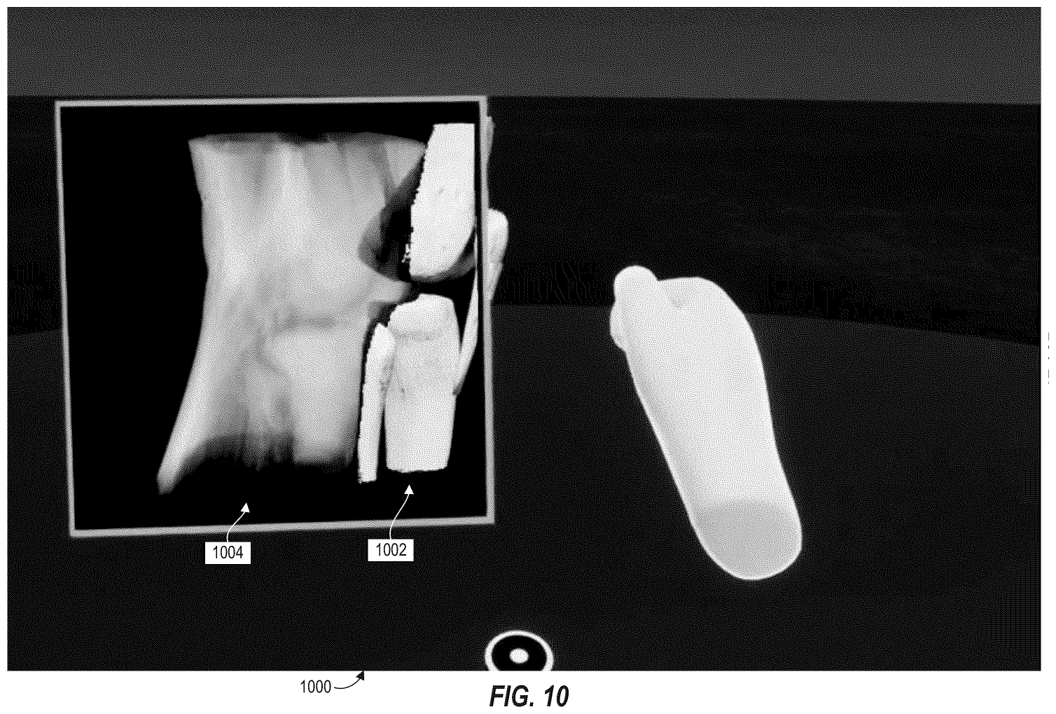

In some embodiments, users may cause idealized 3D representations and subject-specific 3D representations to overlap with one another within a virtual environment, which may provide users with additional comparison paradigms. For example, FIG. 10 illustrates an example virtual environment 1000 in which a user is causing an idealized 3D representation 1002 to spatially overlap with a subject-specific 3D representation 1004. Such functionality can allow users to spatially align idealized representations of bodily structures with subject-specific representations of bodily structures, which can improve user understanding of states of and/or variations in subject-specific bodily structures.

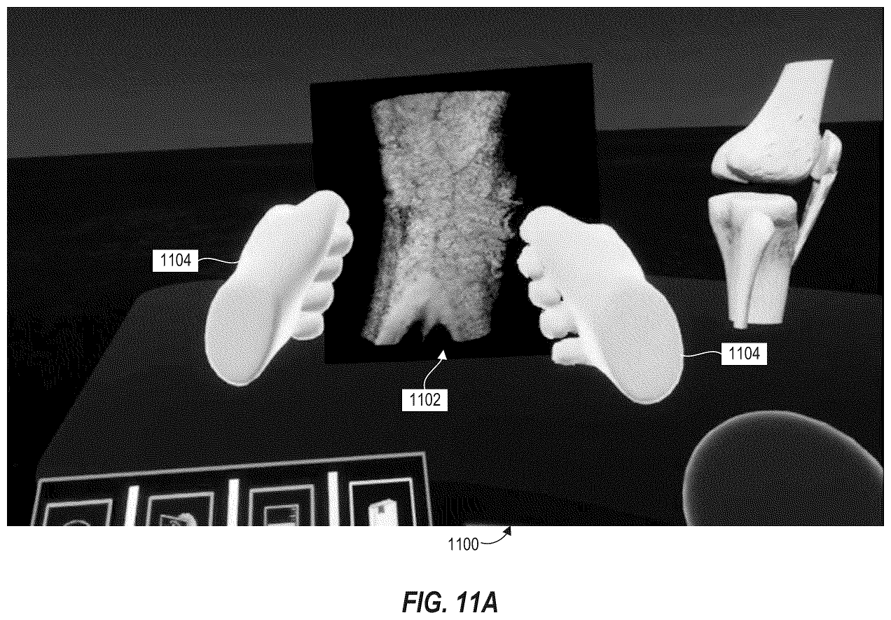

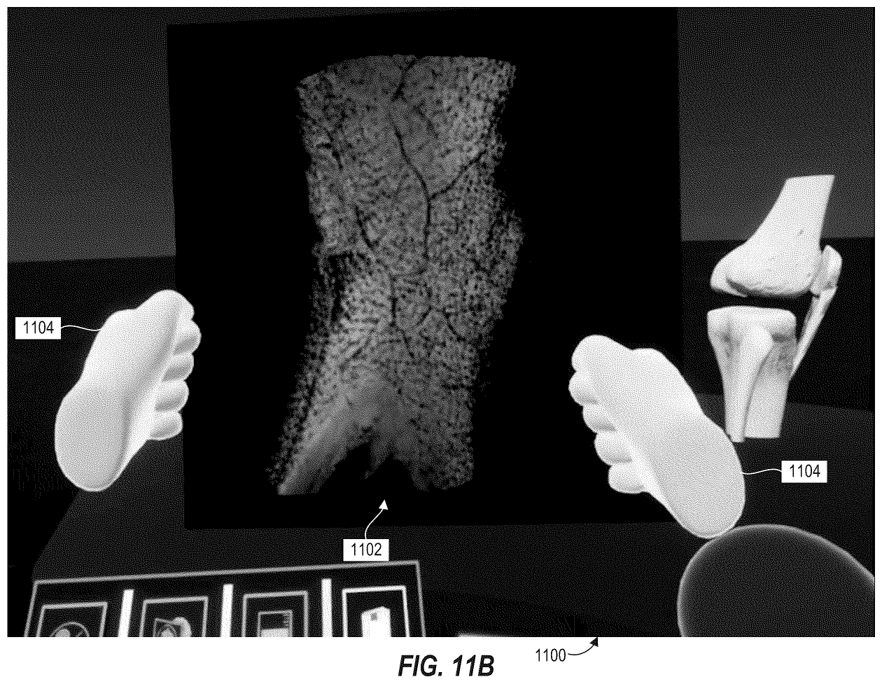

In some implementations, additional types of presentation characteristics (e.g., in addition to position/orientation) of a subject-specific and/or idealized 3D representation may be modified based on user input, such as scale. FIG. 11A illustrates an example instance in which a subject-specific 3D representation 1102 is displayed within a virtual environment 1100 and in which control visualizations 1104 associated with user controls (e.g., the user's hands) are engaged with the subject-specific 3D representation 1102 (e.g., indicated by the pinching or gripping gestures embodied by the control visualizations 1104). Under such a configuration, movement of the control visualizations 1104 (e.g., effectuated by real-world movements of the user's hands or other controllers) may cause modifications to the scale or size with which the subject-specific 3D representation is displayed within the virtual environment 1100. FIG. 11B illustrates an instance in which the subject-specific 3D representation 1102 is presented with different scaling than that shown in FIG. 11A.

One will appreciate, in view of the present disclosure, that other types of user input may be utilized to facilitate modifications to the presentation characteristics (e.g., position, orientation, and/or scale) with which a subject-specific and/or idealized 3D representation is depicted in a virtual environment (e.g., selection of selectable elements that causes the 3D representation(s) to assume predefined presentation characteristics, such as to achieve key frames or key views as the user progresses through an instructional workflow).

In some instances, user input directed to changing presentation characteristics of a subject-specific 3D representation are mapped to facilitate changes to corresponding presentation characteristics of an idealized 3D representation, or vice-versa. Such functionality may be selectively enabled or disabled by the user (e.g., by selectively enabling or disabling a synchronization mode). For instance, when a synchronization mode is active, a user may provide user input to cause modification of the scale, rotation, and/or translation associated with presentation of an idealized 3D representation, and corresponding modifications to the scale, rotation, and/or translation associated with presentation of a subject-specific 3D representation may be automatically implemented. Imposing such view transformations to both 3D representations (e.g., where input is directed to only one of them) may be accomplished in various ways, such as by pre-configuring and/or pre-aligning the subject-specific 3D representation and the idealized 3D representation to a common coordinate space (such that transformation commands on one 3D representation may be readily enacted for both 3D representations by utilizing common coordinates). Such pre-configuring or pre-aligning of the different 3D representations may be accomplished, by way of example, by manual transformation, feature matching, registration markers or points, surface matching, iterative closest point (ICP) algorithms, landmark-based alignment, point cloud registration, and/or other techniques.

Another technique to facilitate imposition of view transformations to both 3D representations can include utilizing pre-generated anatomical labels of structures/components represented in both 3D representations, such that user input directed to a structure/component in one 3D representation may be spatially mapped to a corresponding structure/component in the other 3D representation. For instance, user input directed to using the patella as a zoom center or rotation point in modifying presentation of a subject-specific 3D representation may be mapped to the representation of the patella in the idealized 3D representation to enable the patella in the idealized 3D representation to be simultaneously used as a zoom center or rotation point to modify presentation of the idealized 3D representation. Such functionality, which may be regarded as utilizing anatomical structures as anchor points to facilitate view transformations, may prove beneficial in situations where significant spatial deviations exist in the relative positioning of bodily structure in the subject-specific 3D representation and the idealized 3D representation.

Aside from view transformation inputs, other types of commands/inputs directed to the subject-specific 3D representation may be mapped to or imposed on the idealized 3D representation, or vice-versa. For instance, a user input configured to cause navigation to a particular key frame or key view (e.g., focused on a particular anatomical structure from a particular viewing perspective) for presentation of a subject-specific 3D representation may cause automatic navigation to a corresponding particular key frame or key view for presentation of the idealized 3D representation. Additional types of commands that may be directed to specific anatomical structures and may be mapped to both 3D representations may include visual emphasis, annotation, display/hiding, selecting, sectioning, slicing, and/or others.

Although examples provided herein focus, in at least some respects, on presenting only one idealized 3D representation and/or only one subject-specific 3D representation within a virtual environment, other quantities of idealized 3D representations and/or subject-specific 3D representations may be simultaneously presented, in accordance with implementations of the present disclosure. For instance, in a testing or assessment context, multiple subject-specific 3D representations may be presented in conjunction with a single idealized 3D representation, and a user may be prompted to identify a subject-specific 3D representation in which a particular pathology is present. Furthermore, although examples provided herein focus, in at least some respects, on presenting an idealized 3D representation as a static model, animations may be implemented for the idealized 3D models (and potentially the subject-specific 3D models, depending on the imaging modality). Other functionality may be provided when simultaneously presenting a subject-specific 3D representation with an idealized 3D representation, such as image acquisition tools, audio presentations (e.g., to capture heart palpitations or other anatomical phenomena), and/or others.

Embodiments disclosed herein can include at least those in the following numbered clauses:
Clause 1. A system for facilitating analysis of anatomical structures, the system comprising: one or more processors; and one or more computer-readable recording media that store instructions that are executable by the one or more processors to configure the system to: access a subject-specific 3D representation of one or more anatomical structures, the subject-specific 3D representation being generated based on a set of 2D images of a subject; access an idealized 3D representation of the one or more anatomical structures; and simultaneously display the subject-specific 3D representation and the idealized 3D representation in navigable form within a virtual 3D environment.
Clause 2. The system of clause 1, wherein the set of 2D images comprises a set of grayscale images.
Clause 3. The system of clause 1, wherein the set of 2D images comprises a set of cross-sectional medical images.
Clause 4. The system of clause 1, wherein the system is configured to simultaneously display the subject-specific 3D representation and the idealized 3D representation on a display of an extended reality device.
Clause 5. The system of clause 1, wherein the idealized 3D representation is selected based on one or more attributes of the subject or based on a use context associated with simultaneously displaying the subject-specific 3D representation and the idealized 3D representation.
Clause 6. The system of clause 1, wherein the idealized 3D representation is modified based on one or more attributes of the subject or based on a use context associated with simultaneously displaying the subject-specific 3D representation and the idealized 3D representation.
Clause 7. The system of clause 1, wherein the idealized 3D representation comprises a representation of at least part of an isolated anatomical system.
Clause 8. The system of clause 1, wherein simultaneously displaying the subject-specific 3D representation and the idealized 3D representation comprises visually emphasizing one or more aspects of the one or more anatomical structures represented in both the subject-specific 3D representation and the idealized 3D representation.
Clause 9. The system of clause 8, wherein visually emphasizing the one or more aspects of the one or more anatomical structures represented in both the subject-specific 3D representation and the idealized 3D representation is performed in response to user input directed to the one or more aspects of the one or more anatomical structures in the subject-specific 3D representation or the idealized 3D representation.
Clause 10. The system of clause 1, wherein simultaneously displaying the subject-specific 3D representation and the idealized 3D representation comprises displaying one or more annotations associated with one or more aspects of the one or more anatomical structures represented in both the subject-specific 3D representation and the idealized 3D representation.
Clause 11. The system of clause 10, wherein the one or more annotations are determined based on pre-processing of the subject-specific 3D representation, the set of 2D images, or the idealized 3D representation.
Clause 12. The system of clause 10, wherein the one or more annotations are determined based on user input directed to the one or more aspects of the one or more anatomical structures in the subject-specific 3D representation or the idealized 3D representation.
Clause 13. The system of clause 1, wherein the instructions are executable by the one or more processors to further configure the system to: while simultaneously displaying the subject-specific 3D representation and the idealized 3D representation, receive user input directed to a modification of a presentation characteristic for presenting one of the subject-specific 3D representation and the idealized 3D representation; and in response to the user input, apply the modification of the presentation characteristic for presenting both of the subject-specific 3D representation and the idealized 3D representation.
Clause 14. The system of clause 13, wherein the modification of the presentation characteristic comprises a modification of scale, rotation, or translation.
Clause 15. The system of clause 13, wherein the modification of the presentation characteristic comprises visually emphasizing, annotating, displaying, hiding, selecting, sectioning, or slicing one or more aspects of the one or more anatomical structures.
Clause 16. The system of clause 13, wherein the modification of the presentation characteristic comprises modifying a display filter that constrains display of one or more aspects of the one or more anatomical structures.
Clause 17. A method for facilitating analysis of anatomical structures, the method comprising: causing one or more processors of a system to execute computer-executable instructions stored on one or more computer-readable recording media of the system to configure the system to: access a subject-specific 3D representation of one or more anatomical structures, the subject-specific 3D representation being generated based on a set of 2D images of a subject; access an idealized 3D representation of the one or more anatomical structures; and simultaneously display the subject-specific 3D representation and the idealized 3D representation in navigable form within a virtual 3D environment.
Clause 18. The method of clause 17, wherein the set of 2D images comprises a set of cross-sectional medical images.
Clause 19. The system of clause 17, wherein the system is configured to simultaneously display the subject-specific 3D representation and the idealized 3D representation on a display of an extended reality device.
Clause 20. One or more computer-readable recording media that store instructions that are executable by one or more processors of a system to configure the system to: access a subject-specific 3D representation of one or more anatomical structures, the subject-specific 3D representation being generated based on a set of 2D images of a subject; access an idealized 3D representation of the one or more anatomical structures; and simultaneously display the subject-specific 3D representation and the idealized 3D representation in navigable form within a virtual 3D environment.

### Additional Details Related to Implementing the Disclosed Embodiments

Disclosed embodiments may comprise or utilize a special-purpose or general-purpose computer including computer hardware, as discussed in greater detail below. Disclosed embodiments also include physical and other computer-readable recording media for carrying or storing computer-executable instructions and/or data structures. Such computer-readable recording media can be any available media that can be accessed by a general-purpose or special-purpose computer system. Computer-readable recording media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links that can be used to carry program code in the form of computer-executable instructions or data structures, and which can be accessed by a general-purpose or special-purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that at least some aspects of the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, at least some of the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for facilitating analysis of anatomical structures, the system comprising:
one or more processors; and
one or more computer-readable recording media that store instructions that are executable by the one or more processors to configure the system to:
access a subject-specific 3D representation of one or more anatomical structures, the subject-specific 3D representation being generated based on a set of 2D images of a subject;
access an idealized 3D representation of the one or more anatomical structures; and
simultaneously display the subject-specific 3D representation and the idealized 3D representation in navigable form within a virtual 3D environment.

2. The system of claim 1, wherein the set of 2D images comprises a set of grayscale images.

3. The system of claim 1, wherein the set of 2D images comprises a set of cross-sectional medical images.

4. The system of claim 1, wherein the system is configured to simultaneously display the subject-specific 3D representation and the idealized 3D representation on a display of an extended reality device.

5. The system of claim 1, wherein the idealized 3D representation is selected based on one or more attributes of the subject or based on a use context associated with simultaneously displaying the subject-specific 3D representation and the idealized 3D representation.

6. The system of claim 1, wherein the idealized 3D representation is modified based on one or more attributes of the subject or based on a use context associated with simultaneously displaying the subject-specific 3D representation and the idealized 3D representation.

7. The system of claim 1, wherein the idealized 3D representation comprises a representation of at least part of an isolated anatomical system.

8. The system of claim 1, wherein simultaneously displaying the subject-specific 3D representation and the idealized 3D representation comprises visually emphasizing one or more aspects of the one or more anatomical structures represented in both the subject-specific 3D representation and the idealized 3D representation.

9. The system of claim 8, wherein visually emphasizing the one or more aspects of the one or more anatomical structures represented in both the subject-specific 3D representation and the idealized 3D representation is performed in response to user input directed to the one or more aspects of the one or more anatomical structures in the subject-specific 3D representation or the idealized 3D representation.

10. The system of claim 1, wherein simultaneously displaying the subject-specific 3D representation and the idealized 3D representation comprises displaying one or more annotations associated with one or more aspects of the one or more anatomical structures represented in both the subject-specific 3D representation and the idealized 3D representation.

11. The system of claim 10, wherein the one or more annotations are determined based on pre-processing of the subject-specific 3D representation, the set of 2D images, or the idealized 3D representation.

12. The system of claim 10, wherein the one or more annotations are determined based on user input directed to the one or more aspects of the one or more anatomical structures in the subject-specific 3D representation or the idealized 3D representation.

13. The system of claim 1, wherein the instructions are executable by the one or more processors to further configure the system to:
while simultaneously displaying the subject-specific 3D representation and the idealized 3D representation, receive user input directed to a modification of a presentation characteristic for presenting one of the subject-specific 3D representation and the idealized 3D representation; and
in response to the user input, apply the modification of the presentation characteristic for presenting both of the subject-specific 3D representation and the idealized 3D representation.

14. The system of claim 13, wherein the modification of the presentation characteristic comprises a modification of scale, rotation, or translation.

15. The system of claim 13, wherein the modification of the presentation characteristic comprises visually emphasizing, annotating, displaying, hiding, selecting, sectioning, or slicing one or more aspects of the one or more anatomical structures.

16. The system of claim 13, wherein the modification of the presentation characteristic comprises modifying a display filter that constrains display of one or more aspects of the one or more anatomical structures.

17. A method for facilitating analysis of anatomical structures, the method comprising:
causing one or more processors of a system to execute computer-executable instructions stored on one or more computer-readable recording media of the system to configure the system to:
access a subject-specific 3D representation of one or more anatomical structures, the subject-specific 3D representation being generated based on a set of 2D images of a subject;
access an idealized 3D representation of the one or more anatomical structures; and
simultaneously display the subject-specific 3D representation and the idealized 3D representation in navigable form within a virtual 3D environment.

18. The method of claim 17, wherein the set of 2D images comprises a set of cross-sectional medical images.

19. The method of claim 17, wherein the system is configured to simultaneously display the subject-specific 3D representation and the idealized 3D representation on a display of an extended reality device.

20. One or more computer-readable recording media that store instructions that are executable by one or more processors of a system to configure the system to:
access a subject-specific 3D representation of one or more anatomical structures, the subject-specific 3D representation being generated based on a set of 2D images of a subject;
access an idealized 3D representation of the one or more anatomical structures; and
simultaneously display the subject-specific 3D representation and the idealized 3D representation in navigable form within a virtual 3D environment.
